(19) United States
(12) Patent Application Publication
(10) Pub. No.: US 2008/0312138 A1
(43) Pub. Date: Dec. 18, 2008

(54) Erythropoietin complementation or replacement

(76) Inventors: Claude Nicolau, Newton, MA (US); Jean-Marie Lehn, Strasbourg (FR); Ruth Greferath, Kehl (DE)

Correspondence Address: KING & SPALDING LLP, 1180 PEACHTREE STREET, ATLANTA, GA, 30309-3521, US

(21) Appl. No.: 12/150,946

(22) Filed: May 1, 2008

Related U.S. Application Data
(60) Provisional application No. 60/927,059, filed on May 1, 2007.

(51) Int. Cl. A61K 38/22; A61K 31/7042; A61P 7/06; A61P 7/00

(52) U.S. Cl. 514/8; 514/23

(57) ABSTRACT

The present invention provides methods and compositions to replace up to 90% of erythropoietin use in the treatment of anemias and hypoxias. The method employs acid and salt forms of inositol-tripyrophosphate (ITPP) isomers to shift the P₅₀ value of hemoglobin, thereby improving the rate and efficiency of oxygenation by blood even when red blood cell counts are low. Indications for the new method include anemias and hypoxia arising from infection, chemotherapy, premature birth, altitude change, compromised lung or heart function, aplastic anemia and anemia associated with a myelodysplastic syndrome, and other causes.

### CROSS-REFERENCE TO RELATED APPLICATIONS

This application claims the benefit of U.S. Provisional Patent Application No. 60/927,059, filed May 1, 2007, which is incorporated herein by reference in its entirety.

### FIELD OF THE INVENTION

The present invention is directed to compositions and methods for using the compound inositol-tripyrophosphate (ITPP) to treat anemia. ITPP is an allosteric effector of hemoglobin which has the ability to cross the plasma membrane of red blood cells and lower the oxygen affinity of the hemoglobin of those cells. The present invention is further directed to the use of ITPP as a drug to restore normal oxygenation of red blood cells. The present invention is further directed to the use of ITPP to replace erythropoietin in the treatment of anemia and other associated conditions.

### BACKGROUND OF THE INVENTION

Adult humans have approximately 5 to 6 liters of blood. About one half of this volume is occupied by cells, the majority of which are red blood cells (RBCs, erythrocytes); white blood cells (leukocytes) and blood platelets are also present. Plasma, the liquid portion of blood, is approximately 90 percent water and 10 percent various solutes. These solutes include plasma proteins, organic metabolites and waste products, as well as inorganic compounds.

The major function of RBCs is to transport oxygen from the lungs to other tissues, and to transport carbon dioxide from the tissues to the lungs for removal from the body. Due to the limited solubility of oxygen in aqueous solutions, very little oxygen is transported by blood plasma. Most oxygen carried by blood is bound and transported by the hemoglobin of the erythrocytes. Mammalian erythrocytes contain about 35 percent by weight hemoglobin; they contain no nuclei, mitochondria or other intracellular organelles, and use no oxygen in their own metabolism.

Hemoglobin is a protein having a molecular weight of approximately 64,500 daltons and found exclusively in RBCs. It contains four polypeptide chains and four heme prosthetic groups in which iron atoms are bound in the ferrous state. Normal globin, the protein portion of the hemoglobin molecule, consists of two alpha chains and two beta chains, each with a characteristic tertiary structure of folds and bearing a heme group. The four polypeptide chains fit together in an approximately tetrahedral arrangement, to constitute the characteristic quaternary structure of hemoglobin. Each heme group can reversibly bind one molecule of dioxygen to form oxyhemoglobin; upon release of the oxygen the complex is reduced to deoxyhemoglobin. The four component units of hemoglobin interact with oxygen cooperatively, such that the attractions within alpha-beta dimers are relaxed as oxygen is added, and the fourth oxygen molecule binds to the protein with 300 times more affinity than the first oxygen molecule. By contrast myoglobin, which is a hemeprotein for oxygen transport within heart and skeletal muscle, has a straightforward behavior because it functions much like an isolated single unit of the hemoglobin tetramer.

Delivery of oxygen to tissues depends upon several factors including, but not limited to, the volume of blood flow, number of red blood cells, concentration of hemoglobin in the red blood cells, oxygen affinity of the hemoglobin, and in certain species depends upon the molar ratio of intraerythrocytic hemoglobins with high and low oxygen affinity. The oxygen affinity of hemoglobin in turn depends on four additional factors: (1) the partial pressure of oxygen; (2) pH; (3) concentration of 2,3-diphosphoglycerate (DPG) in the hemoglobin; and (4) concentration of carbon dioxide. In the lungs, at an oxygen partial pressure of 100 mm Hg, approximately 98% of circulating hemoglobin is saturated with oxygen. This represents the entire oxygen transport capacity of the blood. When fully oxygenated, 100 ml of whole mammalian blood can carry about 21 ml of gaseous oxygen.

The effect of oxygen partial pressure on hemoglobin's binding affinity for oxygen is best illustrated by the oxygen saturation curve of hemoglobin, see FIG. 1A. The sigmoidal curve plots the percentage of heme sites that are occupied by oxygen molecules when hemoglobin molecular solutions are in equilibrium over a range of gaseous oxygen partial pressures. Binding the first molecule of oxygen actually increases the oxygen affinity of the remaining open hemoglobin sites. Increasing the partial pressure of oxygen drives the binding affinity toward a plateau at which each hemoglobin is fully saturated with four molecules of oxygen.

The reversible binding of oxygen by hemoglobin is accompanied by release of protons, according to the equation shown below. As illustrated in FIG. 1B, a rise in pH drives the equilibrium to the right and causes hemoglobin to bind more oxygen at a given partial pressure. A fall in pH decreases the amount of oxygen bound. Sources of pH-lowering protons in the blood include carbonic acid formed by the catalyzed reaction of carbon dioxide and water, as well as carbamic acids (—NH—C(═O)—O—H) formed when hemoglobin alpha amine groups bind carbon dioxide for transport.

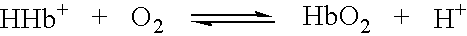

The oxygen partial pressure in lung air spaces is approximately 90 to 100 mm Hg, and the pH is also higher than normal for blood pH (up to 7.6). At that pressure and pH, hemoglobin is approximately 98 percent saturated with oxygen, i.e. near its maximum capacity. By contrast, the partial pressure of oxygen in interior capillaries of peripheral tissues is only about 25 to 40 mm Hg. and the pH there is nearly neutral (about 7.2 to 7.3). Oxygen release is favored in the muscles because those cells use oxygen at a high rate, thereby lowering the local oxygen concentration. Thus, blood passing through muscle capillaries releases about a fourth of its bound oxygen from the nearly saturated erythrocyte hemoglobin into the blood plasma and then into the muscle cells. Hemoglobin is only about 75 percent saturated when it leaves the muscle and, hence, when circulating between the lungs and peripheral tissues, venous blood hemoglobin cycles between about 65 and 97 percent saturation with oxygen. Thus, pH and oxygen partial pressure synergistically affect release of oxygen.

Another important factor in regulating oxygenation of hemoglobin is the allosteric effector 2,3-diphosphoglycerate (DPG). DPG is the normal physiological effector of hemoglobin in mammalian erythrocytes. DPG has an inverse effect: high cellular DPG concentrations lower hemoglobin's affinity for oxygen (see FIG. 1C).

For individuals with chronically low oxygen delivery to the tissues, the ordinary erythrocyte DPG concentration is higher than for the population norm. For example, at high altitudes the partial pressure of oxygen is relatively low so the partial pressure of oxygen in tissues is correspondingly low. Within a few hours after a normal human subject moves to higher altitude the DPG level in red blood cells rises; thus, more DPG is bound and the oxygen affinity of hemoglobin drops, with the result that oxygen is released more easily from RBCs passing through tissues (FIG. 1C). Increases in red blood cell DPG level also occur in patients who suffer from hypoxia; again the adjustment compensates for lower oxygenation of lung hemoglobin. The reverse change occurs when subjects from high altitudes relocate to lower altitudes.

Hemoglobin from normal blood contains a considerable amount of DPG. Hemoglobin that is “stripped” of DPG shows a much higher affinity for oxygen, i.e., its oxygen is released more slowly into tissues. When DPG is increased, the oxygen binding affinity of hemoglobin decreases. Until about six months after birth, humans have a form of hemoglobin, HbF, which binds only weakly to 2,3-BPG and behaves like adult hemoglobin (HbA) that has been stripped of DPG. That characteristic of HbF facilitates the transfer of oxygen from mother to infant across the placenta in the womb, but is problematic for infants who are born significantly prematurely. Outside the womb, it is critically important that hemoglobin have a physiologic allosteric effector such as DPG to facilitate sufficient oxygen release.

Phosphorylated inositols play the same role in some bird and reptile erythrocytes that DPG plays in mammals. Inositol hexaphosphate (IHP) is unable to pass through the mammalian erythrocyte membrane, but can combine with mammalian red blood cell hemoglobin at the binding site of DPG to modify its allosteric conformation, and is far more potent than DPG: IHP has a 1000-fold higher affinity to hemoglobin (R. E. Benesch et al., Biochemistry, 16: 2594-2597 (1977)) and increases the P₅₀ of hemoglobin up to values of 96.4 mm Hg at pH 7.4 and 37 degrees C. (J. Biol. Chem., 250:7093-7098 (1975)).

The enhancement of oxygen release in mammalian RBCs has made allosteric effectors of hemoglobin attractive for treating anemic conditions. Strategies to encapsulate these effectors in erythrocytes have included osmotic pulse (swelling) and reconstitution of cells, controlled lysis and resealing, liposomes, and electroporation.

The following references describe incorporation of polyphosphates into red blood cells by interaction with liposomes loaded with IHP: Gersonde, et al., “Modification of the Oxygen Affinity of Intracellular Hemoglobin by Incorporation of Polyphosphates into Intact Red Blood Cells and Enhanced O₂ Release in the Capillary System”, Biblthca. Haemat., No. 46, pp. 81-92 (1980); Gersonde, et al., “Enhancement of the O₂ Release Capacity and of the Bohr-Effect of Human Red Blood Cells after Incorporation of Inositol Hexaphosphate by Fusion with Effector-Containing Lipid Vesicles”, Origins of Cooperative Binding of Hemoglobin (1982); and Weiner, “Right Shiffing of Hb-O₂ Dissociation in Viable Red Cells by Liposomal Technique,” Biology of the Cell, Vol. 47, (1983).

Additionally, U.S. Pat. Nos. 4,192,869, 4,321,259, and 4,473,563 to Nicolau et al. describe a method whereby fluid-charged lipid vesicles are fused with erythrocyte membranes, depositing their contents into the red blood cells. This allows the transport of allosteric effectors such as IHP into erythrocytes, where IHP's higher binding constant enables displacement of DPG at its hemoglobin binding site.

In the liposome technique, a phosphate buffer solution saturated with IHP is used to suspend a mixture of lipid vesicles, is then treated with ultrasound or an injection process, and centrifuged. The upper suspension has small lipid vesicles containing IHP, which are then collected. Erythrocytes are incubated with the collected suspension, which allows the IHP-containing lipid vesicles to fuse with the cell membranes and deposit their contents into the erythrocyte interior. The modified erythrocytes are then washed and added to plasma to complete the product. Unfortunately, the reproducibility is poor for IHP concentrations incorporated in red blood cells, and significant hemolysis of the cells also occurs following treatment. The procedure is also too tedious and complex for use on a commercial scale.

An attempt to overcome those drawbacks uses a method of lysing and resealing red blood cells. See. Nicolau, et al., “Incorporation of Allosteric Effectors of Hemoglobin in Red Blood Cells. Physiologic Effects,” Biblthca. Haemat., No. 51, pp. 92-107, (1985). Related U.S. Pat. Nos. 4,752,586 and 4,652,449 to Ropars et al. also describe a procedure of encapsulating substances having biological activity in human or animal erythrocytes by controlled lysis and resealing of the erythrocytes, which avoids the red blood cell-liposome interactions. That technique is best characterized as continuous flow dialysis using a technique similar to the osmotic pulse. Specifically, the primary compartment of at least one dialysis element is continuously supplied with an aqueous suspension of erythrocytes, while the secondary compartment of the dialysis element contains an aqueous solution which is hypotonic with respect to the erythrocyte suspension. The hypotonic solution causes erythrocytes to lyse; that lysate is then contacted with the biologically active substance to be incorporated into the erythrocyte. The erythrocyte membranes are resealed by increasing osmotic and/or oncotic pressure of the lysate, and the suspension of resealed erythrocytes is recovered.

U.S. Pat. Nos. 4,874,690 and 5,043,261 to Goodrich et al., disclose a related technique of lyophilization and reconstitution of red blood cells. During that reconstitution step various polyanions, including IHP, are added. Red blood cells treated by the disclosed process are said to have unaffected activity; presumably, the IHP incorporated during reconstitution maintains the hemoglobin activity.

In U.S. Pat. Nos. 4,478,824 and 4,931,276, Franco et al. disclose a comparable approach, the “osmotic pulse technique” and apparatus for introducing effectively non-ionic agents, including IHP, into mammalian red blood cells by effectively lysing and resealing the cells. There a supply of packed red blood cells is suspended and incubated in a solution containing a compound which readily diffuses into and out of the cells, at a concentration sufficient to cause diffusion thereof into the cells so that they become hypertonic. The cellular solution is then diluted with an essentially isotonic aqueous medium in the presence of at least one desired agent to be introduced, so that water diffuses into the cells, causing them to swell and manifest increased permeability in the outer cellular membranes, creating a trans-membrane ionic gradient. The increased permeability is sustained only long enough to transport the desired agent into the cells and diffuse the initial compound out of them.

Polyanions which may be used in practicing the osmotic pulse technique include pyrophosphate, tripolyphosphate, phosphorylated inositols, 2,3-diphosphoglycerate (DPG), adenosine triphosphate, heparin, and polycarboxylic acids which are water-soluble, and non-disruptive to the lipid outer bilayer membranes of red blood cells. Unfortunately, the osmotic pulse technique has several disadvantages, including low yield of encapsulation, incomplete resealing, loss of cellular content and corresponding decrease in cell life span. The technique is tedious, complicated and unsuited to automation; thus, it has had little commercial success.

Another method for encapsulating biologically-active substances in cells is electroporation. Electroporation has been used to encapsulate foreign molecules in various cell types, including IHP in red blood cells, as described in Mouneimne, et al., “Stable rightward shifts of the oxyhemoglobin dissociation curve induced by encapsulation of inositol hexaphosphate in red blood cells using electroporation,” FEBS, 275(1, 2):117-120 (1990). Also, see U.S. Pat. No. 5,612,207. Current methods of electroporation are impractical for use on a commercial scale.

Another method to treat anemia is administration of erythropoietin (EPO), which is a glycoprotein produced naturally in very low levels by the kidneys. It is produced on a commercial scale using recombinant DNA technology in mammalian cell culture, and promotes formation of red blood cells in bone marrow. Commercial names for EPO in its two forms include Epogen®, Eprex®, NeoRecormon®, which are epoetin, and Aranesp®, which is darbepoetin and works in a similar manner. EPO is used to treat anemia from several sources: as a disease or disorder in its own right, as a symptom of another disease such as kidney failure, as cancer-related anemia, and as a side effect of a cancer therapy. See, for instance, Martindale: The Complete Drug Reference (33rd edition). Sweetman et al. Pharmaceutical Press, 2002; British National Formulary (50th edition), British Medical Association and Royal Pharmaceutical Society of Great Britain, September 2005. EPO use has been particularly promising for patients who have anemia associated (chronic) infections such as HIV, inflammatory bowel disease, and septic episodes, and for patients with aplastic anemia and myelodysplastic syndrome.

EPO is commonly used as an alternative to blood transfusions for cancer patients whose hemoglobin levels fall too low due to slowed production of blood cells in bone marrow caused by chemotherapy, and is sometimes supplemented with iron tablets or injections. Red blood cell levels do not begin rising until 2-3 weeks after administration of the compound. EPO is injected subcutaneously, daily if necessary, or as infrequently as every three weeks. The injections usually continue until one month after the chemotherapy course is completed, or until the patient is no longer anemic. EPO doses depend on the indication, but for instance are in the range of ≧300 I.U./kg/week for many cancer patients and renal anemia patients, 100-180 I.U./kg/week for diabetic patients by body weight, and 50 I.U./kg/week for children for some indications.

Common side effects include flu-like symptoms such as joint pains, weakness, dizziness and tiredness, particularly at the beginning of the treatment. A few patients develop severe headaches. High blood pressure can occur. Skin irritation at the injection site or an itchy rash can also occur. EPO use is also associated with an increased risk of adverse cardiovascular complications when it is used to increase hemoglobin levels to levels above 13.0 g/dl. Drüeke T B, Locatelli F, Clyne N, et al., “Normalization of hemoglobin level in patients with chronic kidney disease and anemia,” N. Eng. J. Med., 355(20):2071-2084 (2006). Some trials on EPO benefits have suggested that the compound may in fact facilitate tumor growth. There is also concern that EPO might increase the risk of developing a blood clot (thrombosis).

In March 2007, the US Food and Drug Administration released a Public Health Advisory concerning erythropoietin following a clinical alert to physicians the previous month. The FDA recommended caution in the use of erythropoeisis-stimulating agents such as epoetin and darbepoetin for cancer patients receiving chemotherapy or who were off chemotherapy, citing a lack of clinical evidence to support improvements in quality of life or transfusion requirements in these settings. Also in March 2007, drug manufacturers agreed to new “black box” warnings about the safety of these drugs, and a Congressional inquiry into the safety of erythropoietic growth factors asked manufacturers to suspend those drug rebate programs for physicians and to suspend marketing of the drugs to patients.

Thus, there is an ongoing need for a substantially non-toxic composition and methods that can restore the oxygenation of red blood cells. In particular, there is an ongoing need for a simple and easily administered, preferably oral, composition that can shift the P₅₀ value for red blood cells significantly to the right.

### SUMMARY OF THE INVENTION

It has been discovered that compositions comprising inositol-tripyrophosphate (ITPP) can be used for large-scale replacement of erythropoietin in the treatment of anemias of any type. In the invention method, the use of ITPP assures normal oxygenation even with reduced numbers of red blood cells. Where chemotherapy has slowed or halted erythropoiesis (generation of new red blood cells), as little as 10% of conventional doses of erythropoietin used in the prior art can be used to jump-start the blood cell generation when that treatment is combined with ITPP therapy. Thus, the present invention provides compositions and methods for combination or parallel use of ITPP with EPO, alternation of ITPP with EPO, and replacement of EPO by ITPP, to treat anemias and hypoxia of any type. In particular embodiments, the invention provides a method of treating anemic or otherwise hypoxic humans and animals by replacing up to 90% of prescribed erythropoietin with ITPP administration.

The present invention provides compositions comprising inositol-tripyrophosphate (ITPP) anions that are effective in treating anemias and other hypoxic conditions. The compositions and their use in the present invention have distinct advantages in being substantially non-toxic, causing little if any collateral damage to red blood cells, being essentially free of side effects, providing rapid improvement of oxygenation, and being more easily administered than prior art compositions. The compositions and methods of the invention are also both economically and operationally amenable to use on a commercial scale. In particular embodiments, an ITPP composition is provided in patient-friendly dosage forms.

The present invention also provides methods for increasing the regulated delivery of oxygen to red blood cells by means of ITPP, both within the body and also for blood supplies outside the body. In some embodiments, the invention provides compositions and methods for treating anemia or hypoxia associated with a compromised physiological function. In particular embodiments, the invention provides compositions and methods for preventing or mitigating the hypoxic effects of compromised lung function, compromised heart function, poor circulation, substantial blood loss, loss of or inadequate production of red blood cells, and inadequately oxygenating hemoglobin types.

While not intending to be bound to the following hypothesis, it is believed that ITPP's effectiveness is related to O₂ delivery capacity of red blood cells to hypoxic tissue, increasing the O₂ tension up to the level of normal tissue (i.e., partial pressure ≧˜40 mm Hg). The mechanism of action of ITPP is thought to be enhancement of oxygen release via the allosteric regulation of hemoglobin's affinity for oxygen.

An object of the invention is to provide a substantially non-toxic composition and method for restoring normal oxygenation in humans and animals having anemia and other conditions using ITPP in an effective dose.

Another object of the invention is to provide a composition and method for enhancing oxygen delivery by red blood cells and hemoglobin using ITPP in an effective dose.

Yet another object of the invention is to provide a composition and method for replacing erythropoietin by substituting ITPP in an effective dose.

A further object of the invention is to provide a simple and easily administered, preferably oral, composition using ITPP in an effective dose that is capable of causing significant right shifts of the P₅₀ value for red blood cells on a standard oxygen dissociation curve.

These and other objects, features and advantages of the present invention will become apparent after a review of the following detailed description of the disclosed embodiments.

### BRIEF DESCRIPTION OF THE DRAWINGS

FIG. 1A depicts oxygen dissociation for myoglobin and hemoglobin.

FIG. 1B depicts the effect of pH on the oxygen affinity of hemoglobin.

FIG. 1C depicts the effect of 2,3-BPG on oxygen affinity of hemoglobin.

FIG. 2A tabulates the nature and prevalence of normal adult hemoglobins.

FIG. 2B depicts developmental changes in hemoglobin.

FIG. 3 shows the relationship of P₅₀ shift [%] to number of erythrocytes/mm in mice having received ITPP.

FIG. 4 shows the blood counts of rats treated with doxorubicin or ITPP and of non-treated control rats.

FIG. 5 shows the P₅₀ values and improvement of effort tested in normal wild-type mice.

FIG. 6 demonstrates the improvement of effort capacity in normal wild-type mice after intraperitoneal (ip) injection of 200 μl of a 400 mM and a 150 mM ITPP solution.

FIG. 7 depicts the chemical structure of an exemplary salt of inositol-tri-pyrophosphate (ITPP).

FIG. 8 illustrates the individual differences in the P₅₀ shift induced in the mice by oral ingestion of the aqueous solution of ITPP, versus control animals.

FIG. 9 shows the time course of oral ITPP-induced right shift of the ODC (oxyhemoglobin dissociation curve) P₅₀ in mice, and its absence in control animals.

FIG. 10A shows the time course of the right shift of the ODC in a piglet that received intravenous ITPP, versus a control.

FIG. 10B shows the dosis effect curve of the right shift of the ODC in a piglet that received intravenous ITPP, versus a control.

FIG. 11 shows the dosis effect curve in C57BL/6-mice that received intraperitoneal injections with ITPP.

### DETAILED DESCRIPTION OF THE INVENTION

Compositions that are useful in accordance with the present invention include acids and salts of inositol-tripyrophosphate (ITPP); ITPP is recognized herein as an anion. The term inositol tripyrophosphate, alternatively known as inositol hexaphosphate tripyrophosphate, refers to inositol hexaphosphate with three internal pyrophosphate rings. The counterpart species to ITPP is called a counterion herein, and the combination of ITPP with the counterion is called an acid or salt herein. The invention is not limited to pairings that are purely ionic; indeed, it is well-known in the art that paired ions often evidence some degree of covalent or coordinate bond characteristic between the two components of the pair. The ITPP acids and salts of the invention compositions may comprise a single type of counterion or may contain mixed counterions, and may optionally contain a mixture of anions of which ITPP is one. The compositions may optionally include crown ethers, cryptands, and other species capable of chelating or otherwise complexing the counterions. The compositions may likewise optionally include acidic macrocycles or other species that are capable of complexing the ITPP through hydrogen bonds or other molecular attractions. Methods of making acids and salts of ITPP are described in U.S. Pat. No. 7,084,115 issued to Nicolau et al., the entire content of which is incorporated herein by reference. Counterions contemplated for use in the invention include, but are not limited to, the following:
cationic hydrogen species including protons and the corresponding ions of deuterium and tritium;
monovalent inorganic cations including lithium, sodium, potassium, rubidium, cesium, and copper (I);
divalent inorganic cations including beryllium, magnesium, calcium, strontium, barium, manganese (II), zinc (II), copper (II) and iron (II);
polyvalent inorganic cations including iron (III);
quaternary nitrogen species including ammonium, cycloheptyl ammonium, cyclooctyl ammonium, N,N-dimethylcyclohexyl ammonium, and other organic ammonium cations;
sulfonium species including triethylsulfonium and other organic sulfonium compounds;
organic cations including pyridinium, piperidinium, piperazinium, quinuclidinium, pyrrolium, tripiperazinium, and other organic cations;
polymeric cations including oligomers, polymers, peptides, proteins, positively charged ionomers, and other macromolecular species that possess sulfonium, quaternary nitrogen and/or charged organometallic species in pendant groups, chain ends, and/or the backbone of the polymer.

A particularly preferred isomer for the ITPP employed in the present invention is myo-inositol, which is cis-1,2,3,5-trans-4,6-cyclohexanehexyl; however, the invention is not so limited. Thus, the invention contemplates the use of any inositol isomer in the ITPP, including the respective tripyrophosphates of the naturally occurring scyllo-, chiro-, muco-, and neo-inositol isomers, as well as those of the allo, epi-, and cis-inositol isomers.

It is contemplated that the ITPP may be formed in vivo from a prodrug, such as by enzymatic cleavage of an ester or by displacement of a leaving group such as a tolylsulfonyl group. Use of ITPP generated in this manner for the enhancement of blood cell oxygen economies is considered to be within the scope of the invention.

The term “anemia” as used herein refers to a condition in which the body produces an insufficient number of red blood cells for its oxygen transport needs, or in which the body produces types of hemoglobin which are unable to transport oxygen efficiently in an ambient environment. Examples of the first type of anemia include anemia from the slowing or cessation of blood cell production in bone marrow as a result of chemotherapy, as well as aplastic anemia and anemia associated with a myelodysplastic syndrome. Examples of the latter type of anemia include sickle cell anemia, hemoglobin SC disease, hemoglobin C disease, alpha- and beta-thalassemias, neonatal anemia after premature birth, and comparable conditions.

The term “hypoxia” or “anoxia” are used synonymously herein to refer to a condition in which the tissues of a patient's body receive medically inadequate levels of oxygen. The terms “hypoxia” and “anoxia” as used herein are often coexistent, with but are not coextensive, with anemic conditions.

ITPP is useful in controlling anemia, hypoxia and other associated or related events and conditions, and the invention is not limited by the type of assay used to assess the efficacy of treatment. As used herein, the control of an anemia-associated or related event or condition refers to control evidenced by any qualitative or quantitative change in any type of factor, condition, activity, indicator, chemical species or combination of chemicals, mRNA, receptor, marker, mediator, protein, transcriptional activity or the like, that may be or is believed to be related to anemia, and that results from administering the composition of the present invention. Other such assays include: cell counting in tissue culture plates; assessment of cell number through metabolic assays; and incorporation into DNA of radiolabeled (e.g., by ³H-thymidine) or fluorescently labeled or immuno-reactive (e.g., BrdU) nucleotides.

An erythropoietin treatment regime is defined herein as a therapeutic course of treatment in which the administration of erythropoietin is prescribed at a dosage level and frequency intended to substantially supplement the patient's own natural production of erythropoietin. Erythropoietin as defined herein refers to an erythropoiesis-stimulating agent such as epoetin and darbepoetin, whether derived from natural, manufactured, or recombinant genetic sources. Reduction of an erythropoietin treatment regime refers to the use of smaller doses and or less frequent administrations than the patient had been receiving or than had been prescribed. As defined herein the term reduction of an erythropoietin treatment regime also refers to the use of smaller doses and or less frequent administrations than were commonly reported for the same purposes in patient care and clinical studies up to the end of the year 2006.

Replacement of erythropoietin as defined herein refers to reduction of an erythropoietin treatment regime in combination with the use of another therapeutic agent to compensate in whole or in part for present or prospective oxygenation capacity that is forfeited by reduction of the erythropoietin treatment regime. The present or prospective oxygenation capacity refers to the target efficiency for tissue oxygenation in a patient. Compensation of oxygenation capacity in whole or in part refers to the use of an ITPP composition to preferably replace at least 5% of the existing or hoped-for oxygenation capacity that is forfeited by a reduction in an erythropoietin treatment regime. More preferably, the compensation replaces at least 25% of the oxygenation capacity that is forfeited; still more preferably, it replaces at least 50%; even more preferably, it replaces at least 75%; yet more preferably, it replaces at least 90%; even more preferably, the compensation of ITPP for present (existing) or prospective (hoped-for) oxygenation capacity replaces at least 100% of the capacity that is forfeited by a reduction in an erythropoietin treatment regime.

As defined herein, administration of two compositions in alternating fashion refers to timing the administrations such that in general the body of the patient is estimated to contain therapeutically effective amounts of active material from no more than one of the compositions at any given time. As defined herein, administration of two compositions in parallel refers to administration such that in general the body of the patient is estimated to contain therapeutically effective amounts of active material from both of the compositions at any given time, whether the two compositions are combined into one formulation, or whether the compositions are administered separately in time and as separate formulations, or any combination of the foregoing to achieve the same outcome.

As defined herein, the term PO₂ refers to the partial pressure of oxygen in the gaseous state or in the tissues. As defined herein, the P₅₀ value refers to the equilibrium partial pressure of oxygen in the gaseous state or in the tissues when the available oxygen-binding sites of hemoglobin are 50% occupied by oxygen molecules. As defined herein, a right shift of the P₅₀ value refers to a transformation by which hemoglobin releases oxygen more readily at higher partial pressures of oxygen than had been the case before the transformation. In other words, a right shift of the P₅₀ value refers herein to a decrease in the O₂-affinity of hemoglobin though the PO₂ level remains unchanged.

A substantially low number of red blood cells as defined herein refers to a red blood cell count that is medically deemed to be lower than the healthy normal range for the population. Similarly, a low hematocrit value as defined herein refers to a hematocrit value that is medically deemed to be lower than the healthy normal range for the population.

The effort capacity as defined herein is a measure of a patient's ability to perform physical tasks that are appropriate for the individual's gender, size, weight, and health independent of anemia or hypoxia issues. The effort capacity is an indirect measure of the sufficiency of tissue oxygenation by the patient's red blood cells.

Erythropoiesis, as defined herein, is the generation and reproduction of red blood cells, typically in bone marrow. Slowing or halting of erythropoiesis refers herein to a phenomenon in which a natural, disease-induced or chemically induced deceleration or cessation of erythropoiesis occurs. As defined herein, restarting or jump-starting erythropoiesis refers to the use of an erythropoietic substance such as erythropoietin to accelerate or re-initiate a patient's natural erythropoiesis.

When administered orally, ITPP exhibits anti-anemic activity with little or no toxicity. Myo-ITPP was tested for its ability to induce a decrease of the O₂-affinity of hemoglobin measured as a shift of the P₅₀ value (P₅₀ at 50% saturation of hemoglobin). The observed shifts to higher PO₂ were up to 250% in murine hemoglobin and up to 40% in murine whole blood. This finding was particularly striking because the shifts occurred concomitantly in vivo with a decrease in the number of RBCs and hematocrit; such hemodilution is recognized as a positive indicator in many circumstances because it is diagnostic for downregulation of RBC production where the body's oxygen needs are being met efficiently. Additional support came from enhancement of the effort capacity of test animals by up to 100% following ITPP administration, which confirmed that oxygen was being delivered efficiently to the working muscle, and only to that muscle. In both mice and pigs, the ITPP results strongly support its therapeutic potential, because oxygen delivery by red blood cells can be regulatably enhanced by ITPP during blood flow impairment.

In addition to the compounds of the present invention, the pharmaceutical composition of this invention may also contain, or be co-administered simultaneously or sequentially with, one or more pharmacological agents of value in treating one or more disease or conditions referred to herein. In particular, the invention includes administration of ITPP compositions that include, parallel, alternate, or supplant use of erythropoietin compositions.

A person skilled in the art will be able by reference to standard texts, such as Remington's Pharmaceutical Sciences 17^{th} edition, to determine how the formulations are to be made and how these may be administered.

In a further aspect of the present invention there is provided use of compounds of ITPP, or prodrugs thereof, according to the present invention for the preparation of a medicament for the prophylaxis or treatment of conditions associated with anemia or hypoxia. In a still further aspect of the present invention there is provided a method of prophylaxis or treatment of a condition associated with anemia or hypoxia, said method including administering to a patient in need of such prophylaxis or treatment an effective amount of compounds of ITPP, or prodrugs thereof, according to the present invention, as described herein. It should be understood that prophylaxis or treatment of said condition includes amelioration of said condition.

In a further aspect of the present invention there is provided a pharmaceutical composition comprising compounds of ITPP, or prodrugs thereof, according to the present invention, together with a pharmaceutically acceptable carrier, diluent, adjuvant or excipient. The pharmaceutical composition may be used for the prophylaxis or treatment of conditions associated with anemia or other hypoxia.

By “an effective amount” as referred to in this specification, it is meant a therapeutically or prophylactically effective amount. Such amounts can be readily determined by an appropriately skilled person, taking into account the condition to be treated, the route of administration and other relevant factors. Such a person will readily be able to determine a suitable dose, mode and frequency of administration. “Individual” as referred to in this application refers to any animal that may be in need of treatment for a given condition. “Individual” includes humans, other primates, household pets, livestock, rodents, other mammals, and any other animal(s) that may typically be treated by a veterinarian.

The compositions described above can be provided as physiologically acceptable formulations using known techniques, and these formulations can be administered by standard routes. In general, the combinations may be administered by the topical, oral, rectal, intraperitoneal or parenteral (e.g., intravenous, subcutaneous or intramuscular) route. In addition, the combinations may optionally be incorporated into polymers allowing for sustained release, the polymers being implanted in the vicinity of where delivery is desired, for example, into a cavity or blood vessel that will lead to easy delivery to the place to be treated. The dosage of the composition will depend on the condition being treated, the particular derivative used, and other clinical factors such as weight and condition of the patient and the route of administration of the compound. However, for oral administration, a recommended dosage is in the range of 0.00001 to 10 g/kg/day. A dosage for oral administration is in the range of 0.5 to 2.0 g/kg/day or alternatively, about 0.5 to about 1.5 g/kg/day. In an alternate embodiment, a dosage for oral administration is in the range of about 0.80 to 1.0 g/kg/day or alternatively, about between 0.9 to 1.1 g/kg/day.

The present invention also provides methods for increasing the regulated delivery of oxygen to red blood cells by means of ITPP. In a particular embodiment of the present invention, ITPP is administered orally or internally to restore normal oxygenation of red blood cells in anemia patients. In another embodiment, ITPP is used to treat blood samples prior to transfusions to patients who are or might be anemic or otherwise hypoxic. In another embodiment of the invention, ITPP is used to pre-treat blood samples prior to improve the oxygen releasing capacity prior to transfusions to patients. In a further embodiment, ITPP is used to improve the oxygen economy of blood samples prior to transfusions in order to conserve banked RBCs, especially for rare blood types, while providing the threshold amounts of RBCs to achieve critical oxygenation levels. In yet another embodiment, ITPP is used to treat blood samples during dialysis to improve their oxygen releasing capacity.

In another embodiment, the invention provides a method of treating humans and animals having anemic conditions, by replacing up to 90% of prescribed erythropoietin with ITPP administration.

In another embodiment, the invention provides compositions and methods for mitigating the effect of compromised lung function in humans or animals. In particular exemplary embodiments, the invention provides a method of mitigating damage and improving the comfort and prognosis of patients who suffer from pneumonia, acute or chronic bronchitis, emphysema, pneumoconiosis, coal workers' pneumoconiosis, chronic obstructive pulmonary disease, progressive massive fibrosis, multiple sclerosis, near drowning, toxic vapor inhalation, surfactant inhalation, oily substance inhalation, inadequate lung vasculature, such as in DiGeorge's syndrome, and other conditions that compromise lung function.

In yet another embodiment, the invention provides compositions and methods for preventing or mitigating the effect of a compromised heart function. In particular embodiments these include patients whose hearts have leaky valves, patients who have one or more blocked or mostly blocked arteries, patients whose hearts are stopped or replaced during the course of surgical procedures, and others.

In a further embodiment the invention provides compositions and methods for preventing or mitigating the effect of hypoxia associated with poor circulation. Exemplary indications for this embodiment include diabetes, low blood pressure, and the like.

In still another embodiment, the invention provides compositions and methods for preventing or mitigating the effect of substantial blood loss. Exemplary indications for this embodiment include use with patients who have external injuries, internal bleeding, organ transplants, surgical complications, genetic or drug-related inability to form blood clots, and others.

In additional embodiments, the invention provides compositions and methods for preventing or mitigating the effect of diseases and disorders associated with loss of or inadequate production of red blood cells. Exemplary indications include anemias, such as aplastic anemia and myelodysplastic syndrome, as well as leukemias such as acute myelogenous leukemia, chronic leukemias, and others. Additional exemplary embodiments include use with other indications that require supplementation or replacement of bone marrow.

In still other embodiments, the invention provides compositions and methods for use to improve the oxygen-releasing red blood cell capacity of patients having an inadequately oxygenating hemoglobin type. These embodiments include use for premature infants having substantial amounts of hemoglobin F in their blood, and for patients with hemoglobin disorders, such as sickle cell anemia, hemoglobin C disease, hemoglobin SC disease, alpha-thalassemias and beta-thalassemias.

The formulations in accordance with the present invention can be administered in the form of tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

The formulations include those suitable for oral, rectal, nasal, inhalation, topical (including dermal, transdermal, buccal and sublingual), vaginal, parenteral (including subcutaneous, intramuscular, intravenous, intraperitoneal, intradermal, intraocular, intratracheal, and epidural) or inhalation administration. The formulations may conveniently be presented in unit dosage form and may be prepared by conventional pharmaceutical techniques. Such techniques include the step of bringing into association the active ingredient and a pharmaceutical carrier(s) or excipient(s). In general, the formulations are prepared by uniformly and intimately bringing into association the active ingredient with liquid carriers or finely divided solid carriers or both, and then, if necessary, shaping the product.

Also contemplated by the present invention are implants or other devices comprised of the compounds or drugs of ITPP, or prodrugs thereof, where the drug or prodrug is formulated in a biodegradable or non-biodegradable polymer for sustained release. Non-biodegradable polymers release the drug in a controlled fashion through physical or mechanical processes without the polymer itself being degraded. Biodegradable polymers are designed to gradually be hydrolyzed or solubilized by natural processes in the body, allowing gradual release of the admixed drug or prodrug. The drug or prodrug can be chemically linked to the polymer or can be incorporated into the polymer by admixture. Both biodegradable and non-biodegradable polymers and the process by which drugs are incorporated into the polymers for controlled release are well known to those skilled in the art. Examples of such polymers can be found in many references, such as Brem et al., J. Neurosurg. 74:441-446 (1991), which is herein incorporated by reference in its entirety. These implants or devices can be implanted in a desired vicinity, for example, near the site of new blood cell release from bone marrow, or near lung tissue.

Formulations of the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil emulsion, etc.

A tablet may be made by compression or molding, optionally with one or more accessory ingredients. Compressed tablets may be prepared by compressing, in a suitable machine, the active ingredient in a free-flowing form, such as a powder or granules, optionally mixed with a binder, lubricant, inert diluent, preservative, surface-active or dispersing agent. Molded tablets may be made by molding, in a suitable machine, a mixture of the powdered compound moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutically acceptable carrier. A preferred topical delivery system is a transdermal patch containing the ingredient to be administered.

Formulations for rectal administration may be presented as a suppository with a suitable base comprising, for example, cocoa butter and/or a salicylate.

Formulations suitable for nasal administration, wherein the carrier is a solid, include a coarse powder having a particle size, for example, in the range of 20 to 500 microns which is administered in the manner in which snuff is taken; i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing, in addition to the active ingredient, ingredients such as carriers as are known in the art to be appropriate.

Formulation suitable for inhalation may be presented as mists, dusts, powders or spray formulations containing, in addition to the active ingredient, ingredients such as carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in freeze-dried (lyophilized) conditions requiring only the addition of a sterile liquid carrier, for example, water for injections, immediately prior to use. Extemporaneous injection solutions and suspensions may be prepared from sterile powders, granules and tablets of the kinds previously described.

Formulations contemplated as part of the present invention include nanoparticles formulations made by methods disclosed in U.S. patent application Ser. No. 10/392,403 (Publication No. 2004/0033267) which is hereby incorporated by reference in its entirety. By forming nanoparticles, the compositions disclosed herein are shown to have increased bioavailability. Preferably, the particles of the compounds of the present invention have an effective average particle size of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods well known to those of ordinary skill in the art.

Preferred unit dosage formulations are those containing a daily dose or unit, daily sub-dose, as herein above recited, or an appropriate fraction thereof, of the administered ingredient.

It should be understood that in addition to the ingredients, particularly those mentioned above, the formulations of the present invention may include other agents conventional in the art having regard to the type of formulation in question, for example, those suitable for oral administration may include flavoring agents or other agents to make the formulation more palatable and more easily swallowed.

### EXPERIMENTAL

For the in vitro experiments, ITPP was dissolved in deionized water, pH was adjusted to pH 7 and, for incubation with whole blood, the osmolarity of the ITPP solutions was adjusted with glucose to 270-297 mOsM. Mixtures of hemoglobin and ITPP were measured with a HEMOX analyzer (PD Marketing, London) immediately after mixing. Red blood cells were incubated with ITPP for 1 hour at 37° C. Following incubation, the cells were washed 3 times with Bis-Tris-buffer (pH=7.0) and then used for P₅₀ measurement.

In experiments conducted in vivo in which ITPP was administered orally, ITPP was dissolved in drinking (not deionized) water at a 20 g/L-concentration (=27 mM, pH 7.0) and offered for drinking ad libitum. A significant shift of the P₅₀ value of circulating RBCs was observed.

The following examples illustrate, but do not limit, the invention. Thus, the examples are presented with the understanding that modifications may be made and still be within the spirit and scope of the invention.

### Example 1

### Oral Administration of Tri-Pyrophosphates

Twelve C57BL/6 mice drank ITPP over 4 days (about 25 ml/24 hrs). Seven Control mice drank either pure water (three mice), or a solution of IHP (inositol hexaphosphate without the internal pyrophosphate rings) at the same concentration and pH as ITPP (4 mice). The amount of fluid ingested was the same when offering pure water, IHP-water or ITPP-water, indicating that ITPP-, or IHP-solution was not rejected by the mice. Blood was collected from the tail vein of the 12 C57BL/6 mice on day 0 (before treatment started), 1, 2, 4, 6, 7, 8, 10, 11 and 12, in order to measure P₅₀ values. No C57BL/6 mouse seemed to suffer by this treatment. Oral application of ITPP caused significant right shifts of P₅₀ (up to 31%) in mice.

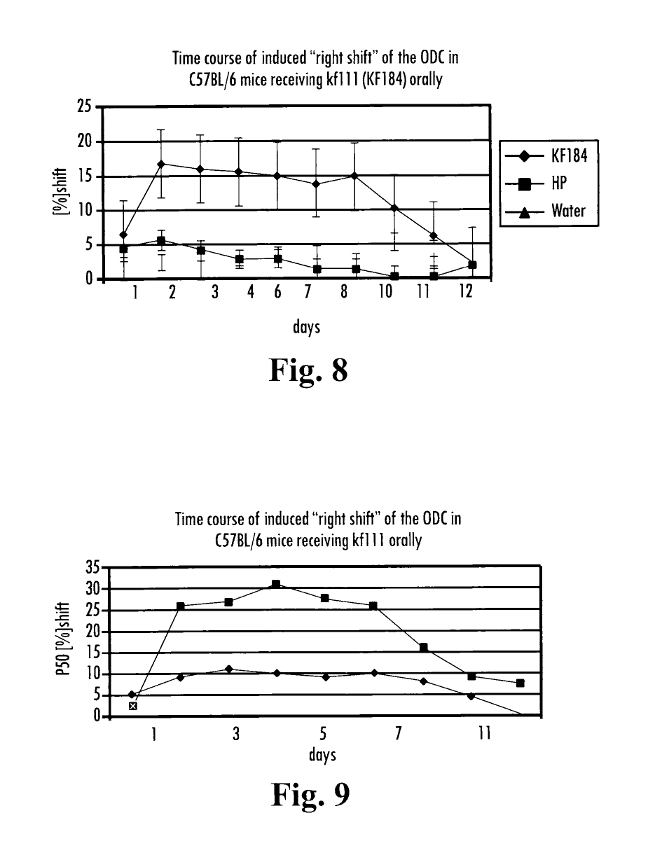

The 12 mice were observed over 12 days, the P₅₀ values of their circulating RBC were measured almost daily. FIG. 9 shows the time course of the induced right shift of the ODC (oxyhemoglobin dissociation curve) P₅₀ (up to 31%) in the mice ingesting ITPP and the complete absence of shift in the control animals ingesting an aqueous solution of IHP or pure water. It appears that all mice ingesting the aqueous solution of ITPP present a shift of the P₅₀ value of their circulating RBC, albeit with individual differences. FIG. 8 illustrates the individual differences in the P₅₀ shift induced in the mice by ingestion of the aqueous solution of ITPP.

### Example 2

### Blood Counts of ITPP-Treated and Control Mice

Blood from mice that ingested ITPP or IHP in water (for 4 days) or water only was collected on day 0, 7 and 11, in order to assess any differences in the blood count (and the amount of erythropoietin in the sera) of treated and control mice. Two major observations were made: 1) the number of RBCs in mice having ingested ITPP was reduced significantly, and 2) there were no major differences in the number of white blood cells (e.g. granulocytes, macrophages etc.) in blood from mice in different groups. Table 1 shows the RBC counts for mice with shifted ODC as compared to controls.

TABLE 1
Number of RBC and P₅₀ shifts of treated and control animals
determined on days 7 and 10 of the experiment

| | P₅₀ | RBC × | P₅₀ | RBC × |
|---|---|---|---|---|
| ITPP | day 7, % | 10⁶/mm³ | day 10, % | 10⁶/mm³ |
| Mouse 1 | 7 | 7.70 | 8 | 8.73 |
| Mouse 3 | 16 | 6.54 | 11 | 7.65 |
| Mouse 4 | 9 | 6.54 | 9 | 7.80 |
| Mouse 5 | 13 | 6.60 | 10 | 9.35 |
| Mouse 6 | 14 | 5.73 | 6 | 8.60 |
| Mouse 7 | 20 | 6.35 | 10 | 8.95 |
| Mouse 8 | 16 | 5.64 | 12 | 8.88 |
| Mouse 11 | 15 | 5.45 | 10 | 8.95 |
| Mouse 12 | 20 | 8.76 | 16 | 8.70 |
| Water | 7 | 9.18 | 12 | 11.35 |
| Water | 4 | 8.7 | 1 | 10.95 |
| IHP | 3 | 9.6 | 0 | 10.77 |

Values of 9 mice that received ITPP, and 2 mice that received water only and 1 mouse that received IHP/water are shown. The amount of blood from the other mice was not sufficient to determine the blood count. (On day 0, the RBC count in the mice was 8.9-11.8×10⁶ cells/mm³). The following conclusions were made from the data.
ITPP, when orally administered at a concentration of 27 mM, causes a significant right shift of the P₅₀ value in murine circulating RBC. A time lag of about 48 hrs occurs before the maximum shift is attained, contrary to observations made after ip inoculation of ITPP, where the P₅₀ shifts appears 2 hrs after inoculation.
Maximal P₅₀ shifts are reached between day 2 and day 4, after beginning oral administration of ITPP.
When ingestion is stopped on day 4, the P₅₀ values return to control values (taken on day 0) within 12 days.
There is a significant effect of ITPP ingestion on the number of RBCs. However, hemolysis of RBCs may be ruled out because lysis of RBCs never occurred in vitro.

It appears that oral administration is effective in shifting the ODC of circulating RBCs in mice, even at modest concentrations of ITPP (27 mM).

### Example 3

### Intravenous Injection of ITPP to a Normal Piglet

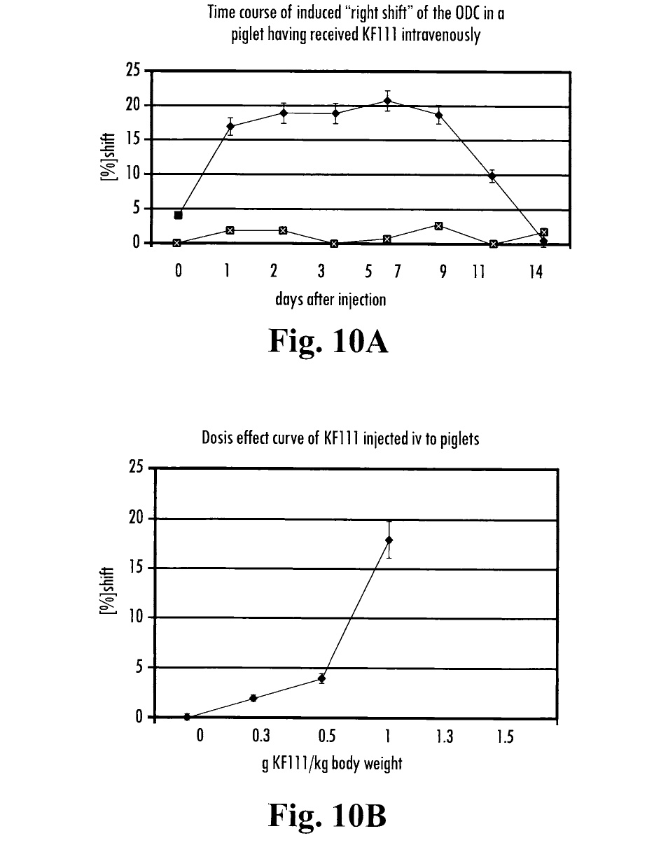

An in vivo experiment was performed on one 8 week-old normal piglet (body weight: 17 kg). The piglet was anesthesized with 5% Isoflurane, 0.7 L/min N₂O and 2.0 L/min O₂ for 20-30 minutes, when ITPP was injected, or blood was taken from the ear vein, respectively. The compound injected intravenous at a concentration of 27 g ITPP/100 ml water (volume injected: 63 ml, pH 6.5, containing 17 g ITPP=1 g/1 kg body weight) was not harmful to the animal, when injected into the piglet's ear vein over at least 10 minutes. The P₅₀ values of the porcine blood obtained during a two-week period after intravenous injection are shown in FIG. 10 versus the control.

### Example 4

### Blood Counts of ITPP-Treated Piglets

Blood from 2 piglets that received ITPP (1 g/kg body weight) was collected before injection, 2 hrs after, and daily over a period of 14 days after injection, in order to assess any differences in the blood counts of treated and non-treated piglets. The following conclusions were drawn:
A slight decrease in hematocrit and in the number of RBCs was observed in the first days after injection.
A tendency towards reduction of the reticulocyte population (from 1.4% to 0.5%) was observed in blood samples collected the first 3 days after injection.
Increasing numbers of reticulocytes were counted in blood samples of the injected animals taken 5-14 days after injection (up to 3.0% on day 14).
Again, no major differences in the number of other cells, such as white blood cells (e.g. granulocytes, macrophages, platelets etc.) were detected.

### Example 5

### Dosis Effect Curves in Piglets and Mice

Intravenous injection of 1 g ITPP/kg body weight caused a significant right shift of the P₅₀-value (up to 20%) in porcine RBCs. An almost saturated ITPP solution, pH 6.7, was injected intravenously into two piglets (both of ca. 18 kg body weight) (27 g ITPP/100 ml=1.5 g/kg body weight) over 20 minutes.

Both piglets died before the injection was completed (at that time point the animals had received <1.3 g/kg body weight=70-80 ml of the saturated ITPP-solution).

Blood was taken from the heart of the dead animals for determination of blood counts as well as the amount of sodium, potassium and calcium in the sera. All numbers of blood cells (hematocrit, white blood cells etc.) were halved. The amount of potassium and calcium was normal, while sodium was doubled (before injection: 120-140 mmol/L; after injection: 245 mmol/L). Apparently, the large amount of sodium in that form of ITPP (6 Na⁺/molecule) caused the death of the animals. It appears that up to 1 g ITPP per kg body weight can be injected intravenously, (if injected slowly) without harmful effects for the animals. The dosis effect curve is shown in FIG. 10B. The following conclusions were drawn from these results:
ITPP was not harmful to the piglet, when applied intravenously slowly (at least 10 min for a vol. of solution of 100 ml)) at a concentration 1 g/kg body weight. A second piglet was also injected with ITPP at the 1 g/kg concentration, after 2 piglets had died after iv injection of 1.2 g ITPP (or even more) per kg body weight. The piglets were thirsty after the treatment.
Higher amounts of ITPP, injected intravenously, killed the animals.
A 1 g ITPP per kg body weight injection is necessary to cause a significant right shift of the P₅₀ value (up to 20%).
Pigs having received this amount of ITPP, at that concentration, did not show any pathological changes of the blood counts, when injected slowly.
In piglets having received 1 g of ITPP/kg body weight, decrease in hematocrit was observed.
No major differences were detectable in the number of white blood cells (e.g. granulocytes, macrophages, platelets, etc.) in blood from the treated piglets.
The number of reticulocytes decreased slightly between 24 and 72 hrs after injection (from 1.5% to 0.5%). Starting with day 3 after injection of the allosteric effector, the number of reticulocytes increased by about 3% for a period of 14 days.

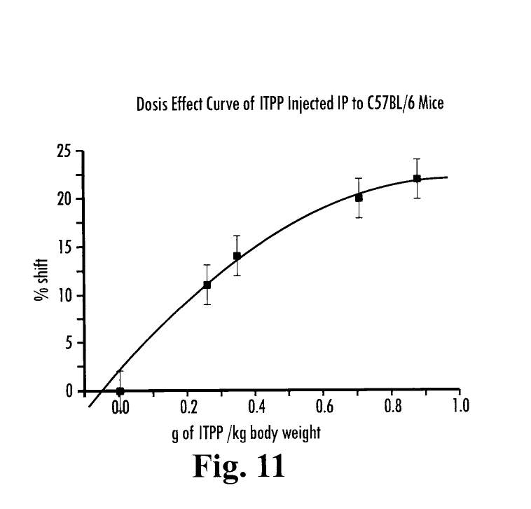

A dosis effect curve was also derived for intraperitoneally (ip) injected ITPP in C57BL/6− mice. Ten mice were injected ip with 45-120 mM of 30 mM ITPP solution. This dosage corresponded to 0.17 to 0.88 g/kg body weight. Six mice were injected with saline solution. FIG. 11 shows the means and the standard deviations observed for the data values in the mice that received ITPP.

### Example 6

### In Vitro Experiments Performed with Whole Blood from Human. Mouse, and Pig

ITPP was tested along with a cholesteroyl derivative (here designated as kf96) (both at 60 mM) as effectors for P₅₀ shifts in whole blood of three species: human, mouse and pig. As usual, pHs for the compound-solutions were adjusted to ca. 7.0, osmolarities for both solutions were determined (325-373 mOsM) prior to treatment with the effectors, and whole blood volumes at 1:1 ratios were incubated. Following incubation, blood cells were washed 3 times with Bis-Tris-buffer; no lysis of RBCs was observed. A summary of P₅₀ values for whole blood induced by the effectors is presented in Table 2.

TABLE 2
P₅₀ values in whole blood after incubation with ITPP and kf96 in vitro*

| | | P₅₀ | | P₅₀ | |
|---|---|---|---|---|---|
| | P₅₀ | mm Hg | P₅₀ | mm Hg | |
| | mm Hg | effector | increase, | effector | P₅₀ |
| Blood | CONTROL | kf96 | % | ITPP | increase, % |
| Human | 22.1 | 28 | 27 | 30.8 | 39 |
| Pig | 32.2 | 41 | 27 | 45.2 | 40 |
| Mouse | 36.7 | 43.9 | 20 | 47.4 | 29 |

*only one animal (or human) for each substance

In all blood samples, a strong right shift in the Hb-O₂ dissociation curve was observed. The shifts obtained with ITPP (up to 40%) were even stronger than with kf96 (27%), and the ITPP is well tolerated by mice even at a concentration of 120 mM.

### Example 7

### Investigation of the Effects of Intraperitoneal Injections of the Effector ITPP

Blood from C57B1/6 mice collected 2 hrs and 1 day after injection of 45, 60, 120 and 150 mM solutions of ITPP was measured for P₅₀-shifts as reported. P₅₀-values of each single sample are listed in Table 3. ITPP was well tolerated even at concentrations of 150 mM. No animal died or seemed to suffer from the compound. There was a shift of P₅₀ at all concentrations, as shown in Table 3.

TABLE 3
P₅₀ values of circulating RBCs after ip-injection of ITPP

| | P₅₀ | | P₅₀ | |
|---|---|---|---|---|
| ITPP | Shift %, | | Shift %, | |
| Concentration | 2 h | Mean +/− SD* | 24 h | Mean +/− SD* |
| 45 mM | 12 | 11.8 +/− 1.16 | 8 | 13.6 +/− 1.02 |
| | 11 | | | |
| | 13 | | | |
| | 10 | | | |
| | 13 | | | |
| 60 mM | 12 | 16.9 +/− 3.48 | 14 | 17.2 +/− 2.1 |
| | 14 | | 16 | |
| | 17 | | 17 | |
| | 21 | | 20 | |
| | 20.5 | | 19 | |
| 120 mM | 28 | 26.0 +/− 2.28 | 28 | 24.8 +/− 2.7 |
| | 29 | | 28 | |
| | 24 | | 22 | |
| | 26 | | 24 | |
| | 23 | | 22 | |
| 150 mM | 26 | 27.0 +/− 1.78 | 25 | 25.8 +/− 2.78 |
| | 28 | | 26 | |
| | 30 | | 31 | |
| | 26 | | 24 | |
| | 25 | | 23 | |

P₅₀ values of blood from 5 animals each are listed;
*SD = standard deviation.

### Example 8

### Relationship of P₅₀ shift [%] to Erythrocyte Population

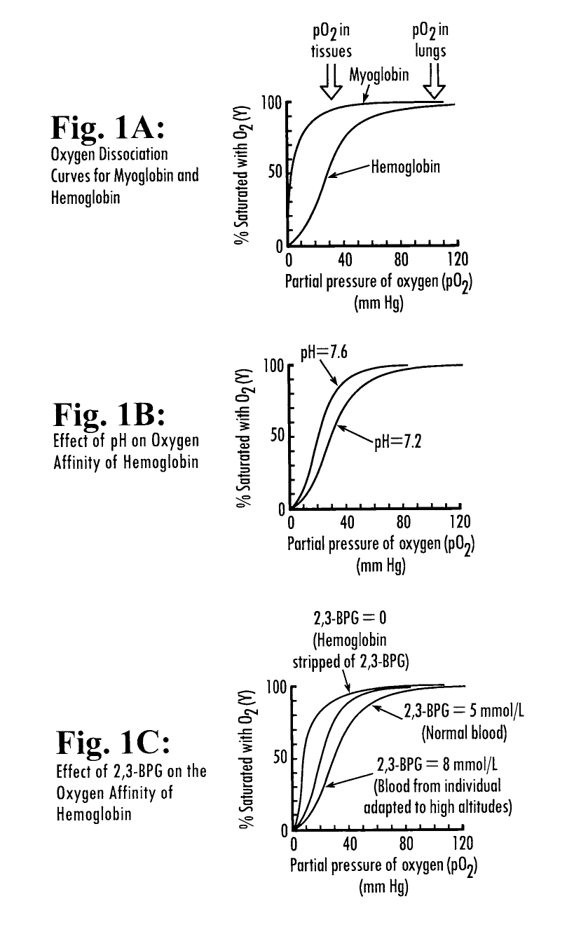

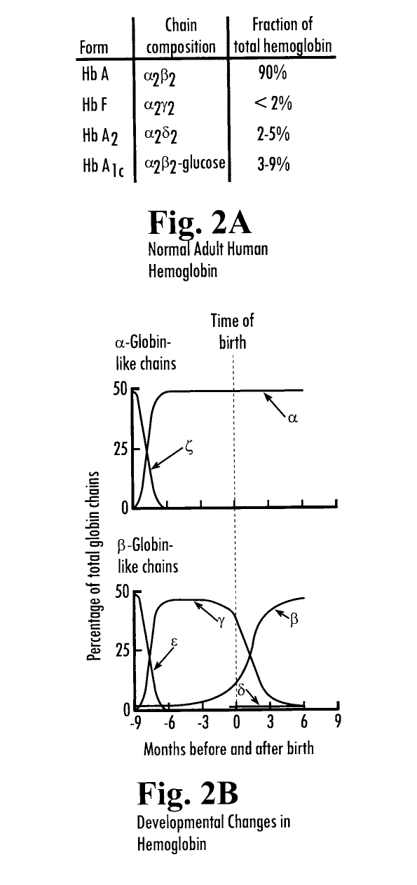

It appears, based upon the preliminary data reported, that an inverse relationship exists between the number of RBCs and shift of their P₅₀ value (see FIG. 1). The basal value of the RBC count is restored, once ΔP₅₀ becomes 0%, 12 days after ingestion of ITPP. The hematocrit drops from 40% on day 0 (before ITPP administration) to 32%, 6 days after IP injection of 200 μl of a 60 mM ITPP solution.

Shifting the P₅₀ value of hemoglobin in circulating red blood cells reduces the number of red blood cells and hematocrit, since fewer red blood cells are needed to oxygenate the organism normally. Thus, hemodilution is a good effect in many circumstances.

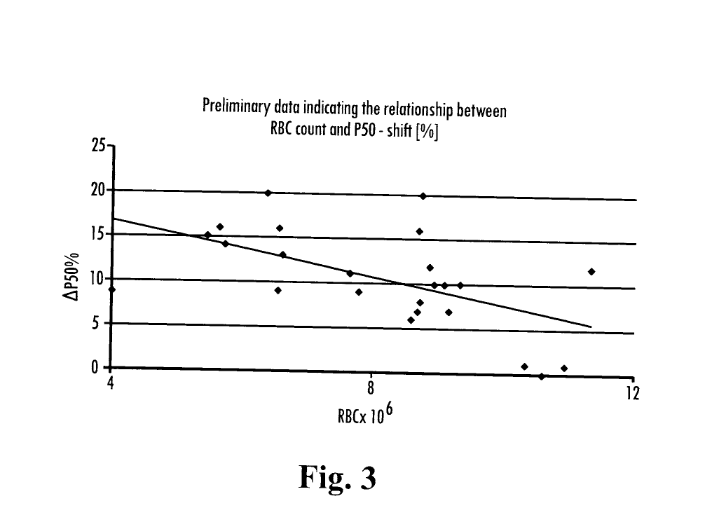

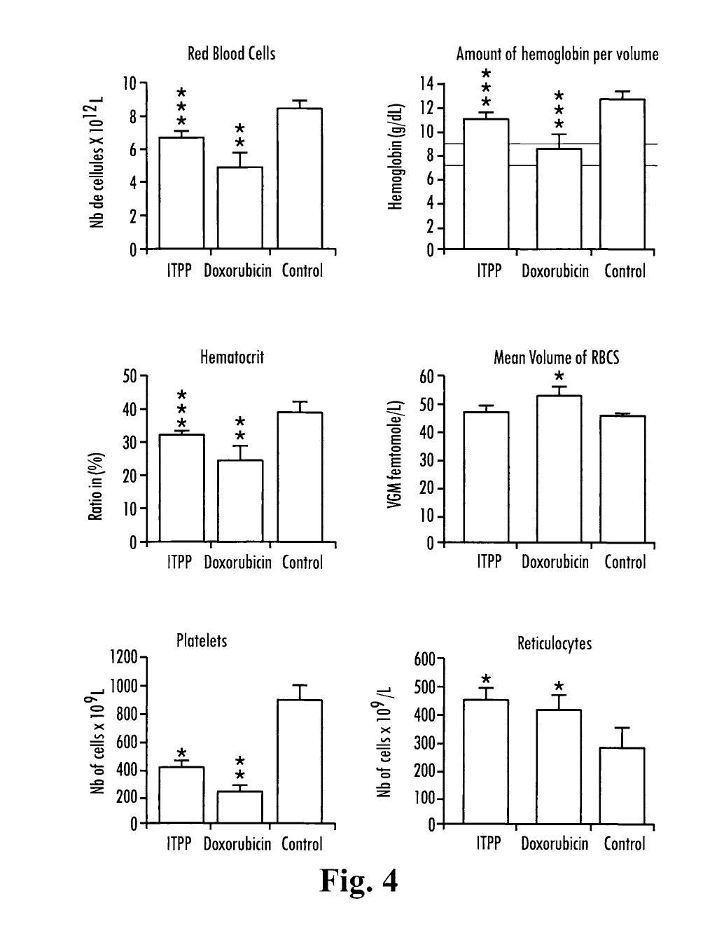

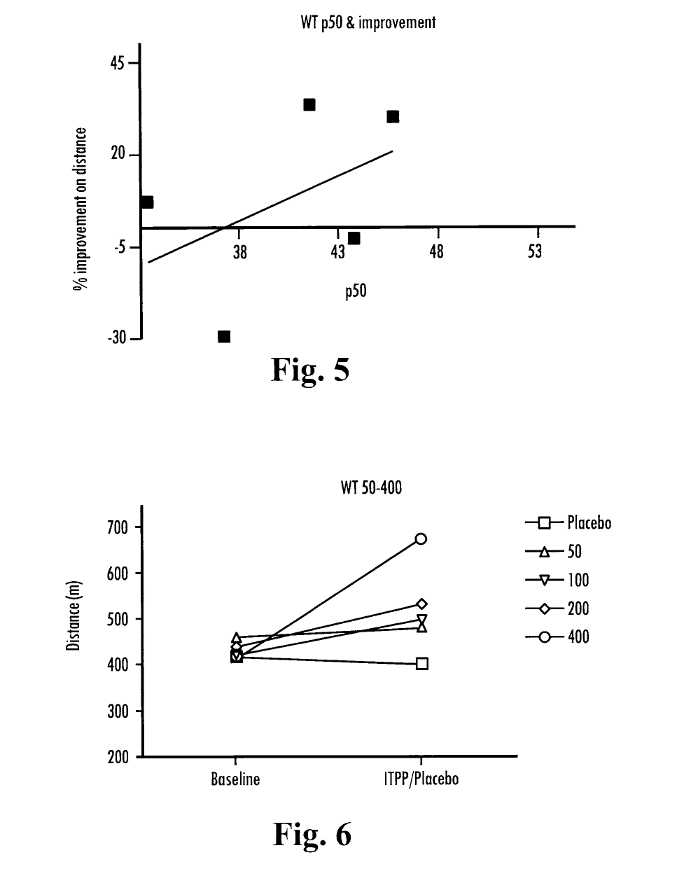

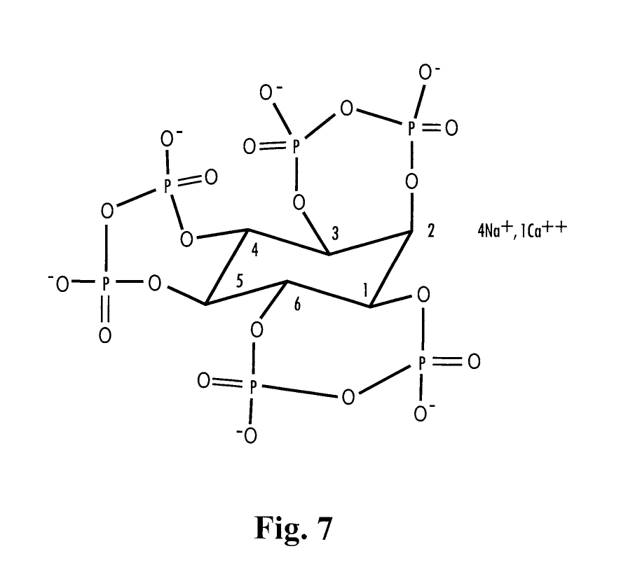

Blood counts are influenced by P₅₀ as shown in FIG. 3, additional proof that ITPP may replace erythropoietin in the treatment of anemias.

### Example 9

### Enhancement of Effort Capacity

The effort capacity of normal animals may be enhanced by up to 100% by ITPP administration, since more oxygen can be delivered to the working muscle. As shown in FIG. 6, a placebo had little effect on distance in meters covered during an effort capacity test of mice, whereas ITPP at a dose of 50 g/kg body weight provided a noticeable improvement, and at 400 g/kg body weight provided about a 70% improvement in effort capacity over the baseline values.

### Example 10

### Preparation of the Calcium Salt of myo-inositol 1.6:2,3:4,5-tripyrophosphate

The hexasodium and hexapyridinium salts of myo-inositol tripyrophosphate (ITPP-Na and ITPP-py) are obtained from myo-inositol hexaphosphate (IHP) as described in K. C. Fylaktakidou, J. M. Lehn, R. Greferath and C. Nicolau, Bioorganic & Medicinal Chemistry Letters, 2005, 15, 1605-1608, which is hereby incorporated by reference in its entirety. Other salts of myo-inositol tripyrophosphate can also be made in accordance with the Fylaktakidou et al. reference. See also, L. F. Johnson and M. E. Tate, Can. J. Chem., 1969, 47, 63, which is also incorporated by reference in its entirety for a description of phytins. And see the syntheses of ITPP acids and salts described in U.S. Pat. No. 7,084,115, issued to Nicolau et al. (Aug. 1, 2006).

Other compounds can be made from the above compounds. For example, passing an aqueous solution of ITPP-py over an ion-exchange Dowex H⁺ column gives a solution of the corresponding perprotonated form of myo-inositol tripyrophosphate (i.e., ITPP-H).

Treatment of the ITPP-H with three equivalents of calcium hydroxide (one equivalent per pyrophosphate group) yields the tricalcium salt ITPP-Ca, which can then be isolated by evaporation of the aqueous solution under reduced pressure such as by use of a rotary evaporator (i.e., a rotovap).

Alternatively, ITPP-Ca can be produced by the addition of equimolar amounts of CaCl₂ with an aqueous solution of ITPP-Na. The resulting mixture gives ITPP-Ca, which contains NaCl as an impurity. It has been found that it is beneficial to have a calcium/sodium mixed salt of ITPP. The pure calcium salt of ITPP was found to be relatively insoluble while the pure sodium salt was found to be relatively more toxic.

Accordingly, in a preferred embodiment, the present invention relates to a calcium salt of inositol tripyrophosphate wherein, optionally, the inositol tripyrophosphate is myo-inositol 1,6:2,3:4,5 tripyrophosphate. It is contemplated that other salts of myo-inositol tripyrophosphate such as the lithium, beryllium, magnesium, potassium, strontium, barium, rubidium and cesium salts of myo-inositol tripyrophosphate can be made and are therefore within the scope of the present invention. These salts can be used in combination with the calcium salt of myo-inositol tripyrophosphate. Alternatively, mixtures of these salts can be made or they can be used without the calcium salt of myo-inositol tripyrophosphate.

In another embodiment, the present invention relates to a pharmaceutical composition comprising the calcium salt of inositol tripyrophosphate and a pharmaceutically acceptable adjuvant, diluent, carrier, or excipient thereof. In this pharmaceutical composition, the inositol tripyrophosphate is optionally myo-inositol 1,6:2,3:4,5 tripyrophosphate. In an alternate embodiment, the composition of the present invention may also optionally contain the sodium salt of myo-inositol tripyrophosphate, preferably in a ratio of 4 Na⁺ ions to 1 Ca⁺⁺ ion per ITPP molecule. It is contemplated and therefore within the scope of the present invention that other myo-inositol tripyrophosphate salts may be used in connection with the calcium salt of myo-inositol tripyrophosphate, including, but not limited to, the pyridinium salt, the N,N-dimethylcyclohexyl ammonium salt, the cycloheptyl ammonium salt, the cyclooctyl ammonium salt, the piperazinium salt and the tripiperazinium salt.

In an embodiment, the above compositions comprise myo-inositol 1,6:2,3:4,5 tripyrophosphate. The composition optionally is prepared at a dosage to treat anemia.

In an embodiment, the composition of the present invention is prepared in any of the above-enumerated ways of delivering a dosage of myo-inositol 1,6:2,3:4,5 tripyrophosphate (such as the calcium salt of this compound) so that between about 0.5 and 1.5 g/kg, and optionally between about 0.9 and 1.1 g/kg per day, is delivered in an effective amount.

In another embodiment, the present invention relates to a method of making the myo-inositol 1,6:2,3:4,5 tripyrophosphate calcium salt wherein the method comprises adding a calcium salt containing organic compound to a perprotonated form of myo-inositol tripyrophosphate. In an embodiment, the calcium salt containing organic compound is one or more of calcium hydroxide, calcium chloride, calcium bromide, calcium iodide, and calcium fluoride. In an embodiment, the method comprises adding at least a three to one ratio of the calcium containing organic compound relative to the perprotonated myo-inositol tripyrophosphate compound amount. Accordingly, in an embodiment, the method comprises adding at least a three to one ratio of the calcium hydroxide relative to the amount of perprotonated myo-inositol tripyrophosphate compound.

In another embodiment, the present invention is related to a method of treating anemia comprising administering to an individual a pharmaceutically acceptable amount of any of the above enumerated compositions, wherein the active ingredient in the composition (i.e., ITPP) is administered to an individual at a dosage of about 0.5 and 1.5 g/kg or alternatively, in an amount that is between about 0.9 and 1.1 g/kg per day.

In an alternative embodiment, the present invention is directed to a method of shifting a hemoglobin P₅₀ level towards higher values of oxygen partial pressure comprising administering to an individual an effective amount of a calcium salt of myo-inositol 1,6:2,3:4,5 tripyrophosphate alone or in combination with one of the above enumerated salts of ITPP. In this method, the calcium salt of myo-inositol 1,6:2,3:4,5 tripyrophosphate optionally is administered as part of a composition wherein the composition optionally contains one or more of an adjuvant, a diluent, a carrier, or an excipient. The calcium salt of myo-inositol 1,6:2,3:4,5 tripyrophosphate in this composition is administered at a dosage of about 0.5 and 1.5 g/kg, or alternatively, at a dosage of between about 0.9 and 1.1 g/kg per day. Alternatively, if other ITPP salts are used in combination with ITPP-Ca, the total dosage of ITPP (from all salt forms and not including the formula weight of the counterions) may be delivered at a dosage of about 0.5 and 1.5 g/kg per day, or alternatively, delivered at a dosage of between about 0.9 and 1.1 g/kg per day.

In another embodiment, the composition of the present invention can be used to treat anemia by delivering an effective amount of an ITPP salt, such as the calcium salt of ITPP.

### Example 11

### Preparation of monocalcium-tetrasodium-myo-inositol-1,6:2,3:4,5-tripyrophosphate

Myo-inositol-1,6:2,3:4,5-tripyrophosphate-H was treated with one equivalent of calcium hydroxide and four equivalents of sodium hydroxide to yield the monocalcium tetrasodium salt composition of ITPP, ITPP-Ca₁Na₄, which is then isolated by evaporation of the aqueous solution under reduced pressure such as by use of a rotary evaporator (i.e., a rotovap).

Alternatively, an ITPP-Ca₁Na₄ composition was produced by the addition of an equimolar amount of CaCl₂ and four equivalents of sodium chloride with an aqueous solution of ITPP-H. The resulting mixture contains HCl as an impurity, which can be removed by rotary evaporation.

It has been found that it is beneficial to have a calcium/sodium mixed salt of ITPP. The pure calcium salt of ITPP was found to be relatively insoluble while the pure sodium salt was found to be relatively more toxic.

### Example 12

### ITPP as a Replacement Therapy for Erythropoietin

In one illustrative example, an erythropoietin treatment regime comprising the administration of 300 I.U. per kg of a patient's body weight per week for treatment of a chemotherapy-induced anemia is reduced to a regime of 30 I.U./kg/week, such that dormant erythropoiesis capacity of the patient may be sustained or revived to prevent or mitigate damage from a chemotherapy treatment. In conjunction with the reduction of the erythropoietin treatment regime, monocalcium-tetrasodium-myo-inositol-1,6:2,3:4,5-tripyrophosphate is administered to the patient as an oral solution at a dosage of between 0.9 and 1.1 g/kg of the ITPP per day.

Having described the invention with reference to particular compositions, method for detection, and source of activity, and proposals of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. It should be understood that any of the above described one or more elements from any embodiment can be combined with any one or more element in any other embodiment. Moreover, when a range is mentioned, it should be understood that it is contemplated that any real number that falls within the range is a contemplated end point. For example, if a range of 0.9 and 1.1 g/kg is given, it is contemplated that any real number value that falls within that range (for example, 0.954 to 1.052 g/kg) is contemplated as a subgenus range of the invention, even if those values are not explicitly mentioned. All references cited herein are incorporated by reference in their entireties.

We claim:

1. A method for enhancing tissue oxygenation by red blood cells in a human or an animal comprising administering to the human or animal a composition comprising an effective amount of inositol-tripyrophosphate (ITPP).

2. The method of claim 1, wherein the ITPP composition further comprises erythropoietin.

3. The method of claim 1, wherein the ITPP composition is used in combination with an erythropoietin treatment regime.

4. The method of claim 1, wherein the ITPP composition is administered in alternating fashion with a second composition comprising erythropoietin.

5. The method of claim 1, wherein the ITPP composition is administered in parallel with a second composition comprising erythropoietin.

6. The method of claim 3 wherein, in any order or simultaneously:
a) the amount of erythropoietin administered to the human or animal is reduced by up to 90% by decreasing the dosage or frequency of administration; and
b) the ITPP composition is administered in a dosage that is calculated to compensate for present or prospective oxygenation capacity that is forfeited by reduction of the erythropoietin dosage.

7. The method of claim 1, wherein the inositol-tripyrophosphate is used as an acid or salt.

8. The method of claim 1, wherein the isomer of inositol in the ITPP composition is selected from the group consisting of myo-, scyllo-, chiro-, muco-, neo, allo-, epi- and cis-isomers of inositol.

9. The method of claim 1, wherein the ITPP composition comprises monocalcium tetrasodium myo-inositol-1,6:2,3:4,5-tripyrophosphate.

10. The method of claim 1, wherein the method is used to shift the P₅₀ value of hemoglobin in circulating red blood cells to the right.

11. The method of claim 1, wherein the method is used to achieve normal oxygenation with a substantially low number of red blood cells.

12. The method of claim 1, wherein the method is used to achieve normal oxygenation at a low hematocrit value.

13. The method of claim 1, wherein the method is used to enhance the effort capacity of the human or animal.

14. The method of claim 1, wherein treatment with the ITPP composition is used to enhance the oxygen carrying capacity of red blood cells that are to be administered to the human or animal, wherein the treatment is performed during hemodialysis or other processing of red blood cells outside the body of the human or animal.

15. A method for treating anemia or hypoxia in a human or an animal comprising administering to the human or animal a composition comprising an effective amount of inositol-tripyrophosphate (ITPP).

16. The method of claim 15, wherein the ITPP composition further comprises erythropoietin.

17. The method of claim 15, wherein the ITPP composition is used in combination with an erythropoietin treatment regime.

18. The method of claim 15, wherein the method is used to treat anemia that is associated with HIV, inflammatory bowel disease, septic episodes, or another chronic infection.

19. The method of claim 15, wherein the method is used in combination with blood transfusions to treat anemia or hypoxia.

20. The method of claim 15, wherein the method is used to prevent or mitigate hypoxia in a human or animal suffering from compromised lung function, compromised heart function, poor circulation, substantial blood loss, an inadequately oxygenating hemoglobin type, or a disease or disorder associated with loss of or inadequate production of red blood cells.

21. The method of claim 15, wherein the inositol-tripyrophosphate is used as an acid or salt.

22. The method of claim 15, wherein the isomer of inositol in the ITPP composition is selected from the group consisting of myo-, scyllo-, chiro-, muco-, neo, allo-, epi- and cis-isomers of inositol.

23. The method of claim 15, wherein the ITPP composition comprises monocalcium tetrasodium myo-inositol-1,6:2,3:4,5-tripyrophosphate.

24. A method for producing erythropoiesis in a human or an animal comprising administering to the human or animal a composition comprising an effective amount of inositol-tripyrophosphate (ITPP).

25. The method of claim 24, wherein the ITPP composition further comprises erythropoietin.

26. The method of claim 24, wherein the ITPP composition is used in combination with an erythropoietin treatment regime.

27. The method of claim 24, wherein the ITPP composition is administered in alternating fashion with a second composition comprising erythropoietin.

28. The method of claim 24, wherein the ITPP composition is administered in parallel with a second composition comprising erythropoietin.

29. The method of claim 26 wherein, in any order or simultaneously:
a) the amount of erythropoietin administered to the human or animal is reduced by up to 90% by decreasing the dosage and or frequency of administration; and
b) the ITPP composition is administered in a dosage that is calculated to compensate for present or prospective oxygenation capacity that is forfeited by reduction of the erythropoietin dosage.

30. The method of claim 24, wherein the inositol-tripyrophosphate is used as an acid or salt.

31. The method of claim 24, wherein the isomer of inositol in the ITPP composition is selected from the group consisting of myo-, scyllo-, chiro-, muco-, neo, allo-, epi- and cis-isomers of inositol.

32. The method of claim 24, wherein the ITPP composition comprises monocalcium tetrasodium myo-inositol-1,6:2,3:4,5-tripyrophosphate.

33. A pharmaceutical composition for treating anemia or hypoxia in a human or an animal comprising inositol-tripyrophosphate (ITPP), and a pharmaceutical carrier or excipient, in an effective amount upon administration in a daily dose, a daily sub-dose, or an appropriate fraction thereof.

34. The pharmaceutical composition of claim 33, wherein the ITPP is monocalcium tetrasodium myo-inositol-1,6:2,3:4,5-tripyrophosphate.

35. A pharmaceutical composition for producing erythropoiesis in a human or an animal comprising inositol-tripyrophosphate (ITPP), and a pharmaceutical carrier or excipient, in an effective amount upon administration in a daily dose, a daily sub-dose, or an appropriate fraction thereof.

36. The pharmaceutical composition of claim 35, wherein the ITPP is monocalcium tetrasodium myo-inositol-1,6:2,3:4,5-tripyrophosphate.
